(19) United States
(12) Patent Application Publication
(10) Pub. No.: US 2014/0005595 A1
(43) Pub. Date: Jan. 2, 2014

(54) DELIVERY DEVICES AND METHODS FOR DELIVERING THERAPEUTIC AGENTS

(71) Applicants: W. Robert Taylor, Stone Mountain, GA (US); Raymond P. Vito, Smyrna, GA (US); Michael L. Wach, Alpharetta, GA (US); Jack Griffis, Decatur, GA (US)

(72) Inventors: W. Robert Taylor, Stone Mountain, GA (US); Raymond P. Vito, Smyrna, GA (US); Michael L. Wach, Alpharetta, GA (US); Jack Griffis, Decatur, GA (US)

(73) Assignees: CELL PRECISION, LLC, Smyrna, GA (US); EMORY UNIVERSITY, Atlanta, GA (US)

(21) Appl. No.: 14/004,738

(22) PCT Filed: Mar. 19, 2012
(86) PCT No.: PCT/US12/29648

§ 371 Date: Sep. 12, 2013

Related U.S. Application Data
(60) Provisional application No. 61/453,697, filed on Mar. 17, 2011.

(51) Int. Cl. A61M 31/00

(52) U.S. Cl. CPC A61M 31/007
USPC 604/60

(57) ABSTRACT

Devices and methods for delivering therapeutic agents use a movable sheath member to deliver a therapeutic agent with little or no shear stress. The delivery device may include an outer body being configured to move away from a distal end of the delivery device. The movable sheath member may have a first section and a second section opposing the first section, the second section being configured to hold the therapeutic agent. The movable sheath may be configured to deliver the therapeutic agent by increasing the first section by moving the outer body away from the distal end.

### CROSS REFERENCE TO RELATED APPLICATION

This application claims priority to Provisional Application Ser. No. 61/453,697 filed Mar. 17, 2011, which is hereby incorporated by reference in its entirety.

### BACKGROUND

An emerging approach to treating disease entails encapsulating a therapeutic agent in a casing to form beads, capsules, or similar structures that a medical practitioner places in a patient adjacent diseased tissue. This approach retains the therapeutic agent at the treatment site and focuses the therapeutic agent's effect on the diseased tissue. For example, beads formed of therapeutic cells, such as stem or progenitor cells, encased in an alginate can be delivered to a strategic location of a patient's leg to treat peripheral artery disease. The casing functions as a membrane across which materials transfer to and from the therapeutic agent payload. The casing's permeability beneficially limits the sizes of the materials that can pass into or out of the bead's interior.

Conventional technologies for delivering such beads, and other systems carrying therapeutic agent payloads, generally lack sufficient sophistication for realizing full therapeutic potential. Aggressive handling resulting from these technologies, such as attempting to inject the beads with a conventional syringe, may compromise therapeutic effectiveness due to shear forces, bead-to-bead impact, and/or imprecise placement at a treatment site. Accordingly, a need exists for improved delivery methods and devices.

### SUMMARY

The disclosure relates to methods and delivery devices for delivering a therapeutic agent. These methods and delivery devices delivery a therapeutic agent with little or zero shear because the relative position of the therapeutic agent within the delivery device does not change while delivered.

In some embodiments, the disclosure relates to a delivery device for delivering a therapeutic agent from a distal end. The delivery device may include: an outer body being configured to move away from the distal end; and a movable sheath having a surface that includes a first section and a second section. The first section may overlap a portion of the second section, the second section being configured to hold a therapeutic agent; the second section may surround an inner cavity configured to hold a therapeutic agent; and the movable sheath may be configured to deliver the therapeutic agent by increasing the first section by moving the outer body away from the distal end.

In some embodiments, an end of the movable sheath may be fixedly attached to a portion of the outer body. In other embodiments, the movable sheath may be configured to form the distal end. In further embodiments, the movable sheath may be configured to extend about the distal end. In other embodiments, the movable sheath may be configured to deliver the therapeutic agent by increasing the first section by inverting a portion of the second section.

In some embodiments, the movable sheath may include a plurality of perforations; and the perforations may be configured to split when the first section is increased.

In other embodiments, the delivery device may comprise: a push rod that is configured to move toward the distal end; an outer body that is configured to move away from the distal end; and a movable sheath including a first section and a second section that opposes a portion of the first section, the second section surrounding an inner cavity configured to hold and deliver the therapeutic agent. In some embodiments, the outer body may be concentrically arranged about the second section.

In further embodiments, the delivery device may include a main body concentrically disposed between the outer body and the movable sheath, the main body extending between the outer body and the push rod. In some embodiments, the outer body and the push rod may be configured to move with respect to the main body. In some embodiments, the movable sheath may be configured to deliver the therapeutic agent by moving the push rod toward the distal end and the outer body away from the distal end.

In other embodiments, the main body may include a first surface and a second surface that opposes the first surface and borders a cavity, the first section extending along the first surface and the second section extending along the second surface. In some embodiments, the movable sheath may be configured to increase the first section and decrease the second section when the outer body and the push rod are moved. In other embodiments, the movable sheath may have a first end and a second end, the first end being fixedly disposed to an end of the push rod and the second end being fixedly disposed to an end of the outer body.

In other embodiments, the delivery device may include: a movable sheath having a surface, the surface includes a first section and a second section that overlaps a portion of the first section, the second section surrounding an inner cavity configured to hold and deliver the therapeutic agent, a fixed rod; and an outer body concentrically arranged about a portion of the movable sheath and the fixed rod, wherein the outer body is configured to move toward the fixed rod. In some embodiments, the movable sheath may be configured to deliver the therapeutic agent by moving the outer body away from the distal end. In other embodiments, the movable sheath may be configured to increase an overlap between the first section and the second section when the outer body is moved.

In some embodiments, the movable sheath may be configured to deliver a therapeutic agent when the first section is increased. In further embodiments, the surface of the movable sheath may be configured to move in a first direction and a second direction that is opposite of the first direction. In other embodiments, the first direction may be towards the distal end, and the movable sheath may be configured to deliver a therapeutic agent when the surface changes from the first direction to the second direction. In some embodiments, the outer body may be configured to expose the movable sheath when the outer body is moved away from the distal end.

In other embodiments, the delivery device may have a length and may include a movable sheath, the delivery device being configured to deliver the therapeutic device when a length of the delivery device is shortened.

In further embodiments, the disclosure relates to a method of delivering a therapeutic agent. The method may comprise positioning a movable sheath holding a therapeutic agent adjacent to a treatment site; causing a section of the movable sheath to invert; and releasing the therapeutic agent from the movable sheath.

In some embodiments, the method may comprise: pulling a outer body that engages a movable sheath while pushing a push rod, the movable sheath having a surface, the surface having a first section and a second section opposing the first section, the second section surrounding an inner cavity that is configured to hold the therapeutic agent; the pulling causing at least a portion of the second section to invert along a length of a main body; and delivering the therapeutic agent as the portion of the second section inverts.

In other embodiments, the method may comprise: pulling an outer body that engages a movable sheath while holding a fixed rod, the inverting member having a movable continuous surface, the movable sheath having a surface, the surface having a first section and a second section opposing the first section, the second section surrounding an inner cavity that is configured to hold the therapeutic agent; the pulling causing at least a portion of the second section to invert and the first section and the portion of the second section to be exposed; and delivering the therapeutic agent as the portion of the second section inverts.

Additional advantages of the disclosure will be set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DESCRIPTION OF THE DRAWINGS

The disclosure can be better understood with the reference to the following drawings and description. The components in the figures are not necessarily to scale, emphasis being placed upon illustrating the principles of the disclosure.

FIG. 1 shows a delivery device according to an embodiment;

FIG. 2 is a front view of the delivery device shown in FIG. 1;

FIG. 3 is a side view of the delivery device shown in FIG. 1;

FIG. 4 is a cross-sectional view of the delivery device taken along the line 4-4 of FIG. 3;

FIG. 5 is an enlarged partial view of the cross-sectional view of FIG. 4;

FIG. 6 illustrates the delivery device shown in FIG. 1 in a state of operation;

FIG. 7 is a side view of the delivery device shown in FIG. 6;

FIG. 8 is a cross-sectional view of the delivery device taken along the line 8-8 of FIG. 7;

FIG. 9 illustrates the delivery device shown in FIG. 1 in another state of operation;

FIG. 10 is a side view of the delivery device shown in FIG. 9;

FIG. 11 is a cross-sectional view of the delivery device taken along the line 11-11 of FIG. 10;

FIG. 12 shows a delivery device according to another embodiment;

FIG. 13 is a front view of the delivery device shown in FIG. 12;

FIG. 14 is a side view of the delivery device shown in FIG. 12;

FIG. 15 is a cross-sectional view of the delivery device taken along the line 15-15 of FIG. 14;

FIG. 16 is an enlarged partial view of the cross-sectional view of FIG. 15;

FIG. 17 illustrates the delivery device shown in FIG. 12 in a state of operation;

FIG. 18 is a side view of the delivery device shown in FIG. 17;

FIG. 19 is a cross-sectional view of the delivery device taken along the line 19-19 of FIG. 18;

FIG. 20 illustrates the delivery device shown in FIG. 12 in another state of operation;

FIG. 21 is a side view of the delivery device shown in FIG. 20; and

FIG. 22 is a cross-sectional view of the delivery device taken along the line 22-22 of FIG. 21.

### DESCRIPTION OF THE EMBODIMENTS

The following description, numerous specific details are set forth such as examples of specific components, devices, methods, etc., in order to provide a thorough understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

It will be understood that the delivery devices according to the embodiments may be implanted into a patient with use of a delivery system. The delivery devices may be a part of the delivery system. The proximal end (not shown in detail) of the disclosed delivery devices may be attached or connected to the delivery system. The disclosed delivery devices may be either part of the main body of the delivery system or may be a detachable component, such as a cartridge. The disclosed delivery devices may be attached to the distal end of a delivery system for insertion into a patient. The delivery system may be any known delivery system, including, but not limited to, a trocar or a cannula, or a flexible catheter. It will also be understood that a clinician may use a delivery system to control the operation of the disclosed delivery devices. It will be further understood that although the operations of the disclosed delivery devices are discussed with respect to the actions of a clinician, delivery systems may be configured to perform these actions.

The delivery systems and the disclosed devices may be adjusted and sized for the anatomy and the direction of the desired point of insertion. For example, the delivery systems and devices may be sized for lengthwise insertion into a vascular lumen. The disclosed delivery devices may also be adjusted according to the requirements of the therapeutic agent(s) to be delivered to the patient. The requirements may include, but not limited to, the size and the dosage amount of the therapeutic agent(s).

The terms “distal” and “proximal” used herein with respect to the delivery device and features are with respect to the position of the delivery device when in use. “Distal” indicates an end of the delivery device or a feature of the device closest to, or a direction towards the treatment site, and “proximal” indicates an end of the device or a feature of the device farthest from, or a direction away from the treatment site. “Treatment site” refers to any site on a subject, human or animal, intended to be treated, such as a diseased tissue.

The delivery devices and systems according to embodiments may be configured to deliver a therapeutic agent(s). The therapeutic agent(s) may include any one or more substance, compound, composition, formulation, and/or agent capable of exerting an effect, such as an therapeutic, prophylactic or diagnostic effect, on a patient. The therapeutic agent(s) may be embedded or dispersed in alginate strings or filaments, encapsulated, suspended in a highly viscous solution, suspended in a high-volume fraction solution, as well as provided in any known vehicle to deliver a therapeutic agent. Such strings or filaments may be end loaded into the distal end of the delivery devices and systems according to embodiments, without necessarily feeding from the proximal end to the distal end. Suitable techniques for making therapeutic agent(s), for example, making strings and filaments comprising cells and alginate, are known in the art and may be readily created by those of ordinary skill having benefit of the present teaching. See, for example, “Grafting microcapsules of genetically modified cells: Therapeutic potential in spinal cord injury;” M. Wheatley, N. Dhoot, S. Kanakasabai and I. Fischer; Drexel University, Philadelphia, USA; XVth International Workshop on Bioencapsulation, Vienna, Au.; Sep. 6-8, 2007; S7-2, pages 1-4.

According to embodiments, a delivery device may include a movable peel-away sheath to deliver a therapeutic agent. A delivery device according to the embodiments may reduce or eliminate the shear stress associated with the delivery of the therapeutic agent because the agents move with respect to the delivery device and not with respect to the sheath, itself A delivery device according to the embodiments may be configured to also allow for the advancement of the therapeutic agent into the site of the local delivery with minimal difficulty for the clinician or discomfort for the patient. A delivery device according to the embodiments thus may reduce the difficulty with delivering high viscosity therapeutic agents.

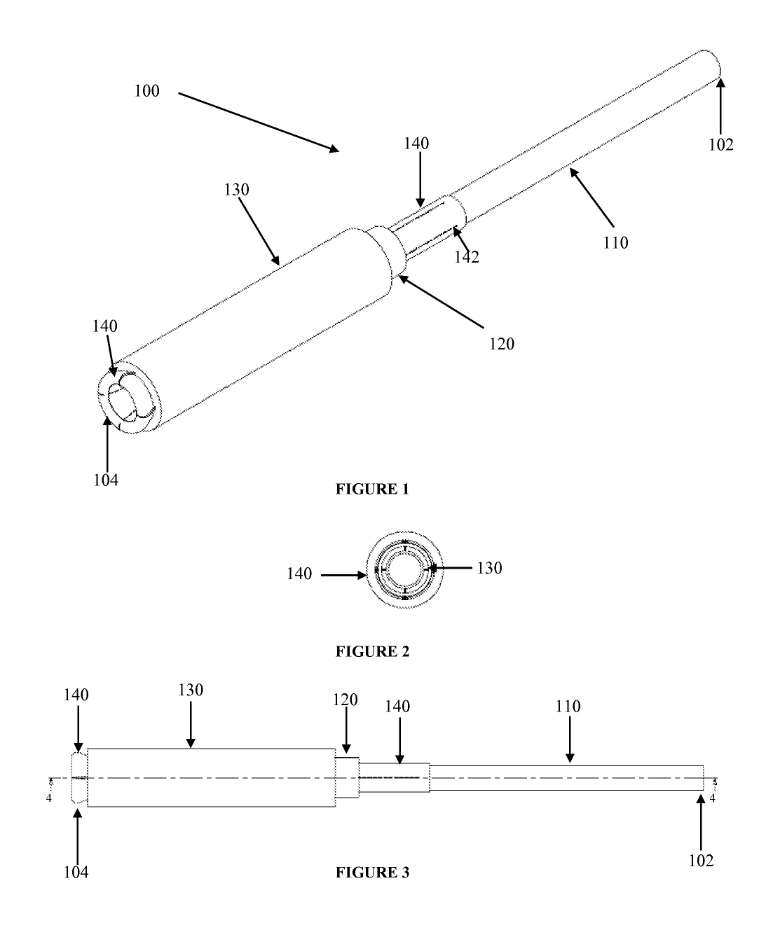

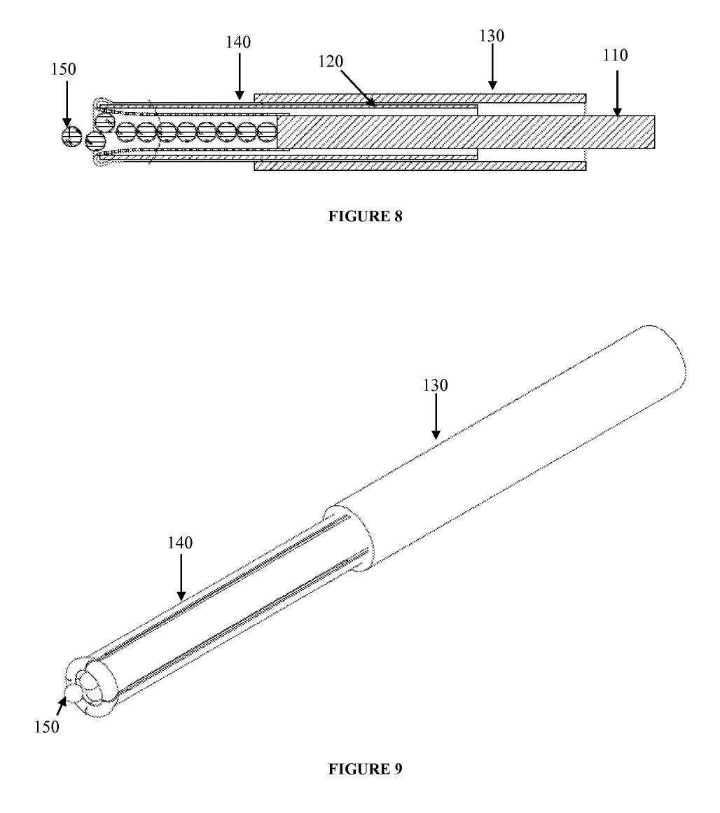

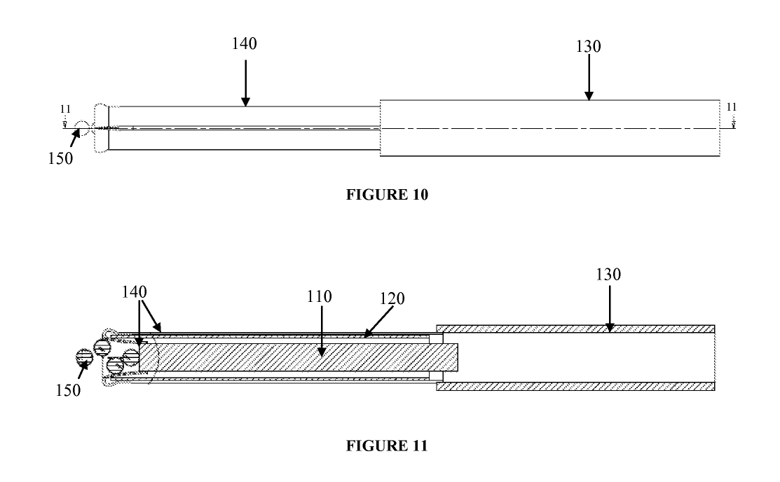

According to some embodiments, a delivery device may be configured so that a peel-away sheath is exposed while delivering a therapeutic agent. In some embodiments, the ends of the sheath may be fixedly attached to an outer surface of a main body or a retraction channel, and thus, during retraction, expose and release the contents. FIGS. 1 through 11 illustrate a delivery device according to these embodiments.

As shown in FIGS. 1 through 4, a delivery device 100 may include a push rod 110, a main body 120, an outer body 130, and a movable peel-away sheath (hereinafter referred to as “movable sheath”) 140.

The push rod 110 may be a straight piece of material. The push rod 110 may be any shape, including but not limited to, round or square. The diameter of the push rod 110 may be mated with the inner cavity of the main body 120 (discussed in more detail below). The push rod 110 may be configured to move towards a distal end 104 of the delivery device 100 through the main body 120.

The main body 120 may have a tubular structure. The main body 120 may be concentrically arranged about a section of the movable sheath 140 and a portion of the push rod 110. The main body 120 may include a first surface 122 and a second surface 124. The second surface 124 may be the interior surface that borders an inner cavity 126. The movable sheath 140 may extend along portions of the first surface 122 and of the second surface 124. In some embodiments, the main body 120 may be configured to be immovable with respect to the delivery device 100.

The outer body 130 may also have a tubular structure. The outer body 130 may be concentrically arranged about both the main body 120 and the movable sheath 140.

The push rod 110, the main body 120, and the outer body 130 may be made of a biocompatible high strength material. The push rod 110, the main body 120, and the outer body 130 may be made of the same or different material. In some embodiments, the material may be surgical stainless steel, for example, 300 series. In other embodiments, the material may be liquid crystal polymer tubing.

The movable sheath 140 may be a circular surface. In some embodiments, at least one surface of the movable sheath 140 may be configured to invert. The movable sheath 140 may include a first section 142 and an overlapping or opposing second section 144. The first section may 142 may extend from a portion of the push rod 110 along of the length of the moving body 120, which includes a length of the outer body 130, through the distal end 104. The first section 142 may extend along a portion of the first surface 122. The second section 144 may extend from the distal end 104 along a length of the moving body 120, which includes a length from the outer body 130. The second section 144 may extend along a portion of the second surface 124.

The movable sheath 140 may be made of a biocompatible material having a low surface coefficient of friction. In some embodiments, the movable sheath 140 may be made of a plastic having a low surface of coefficient of friction. Examples of such a plastic include, but are not limited to, polytetrafluoroethylene (also known as Teflon®), fluorinated ethylene propylene (FEP), perfluoroalkoxy (PFA). In other embodiments, the movable sheath may be made of polyethylene.

In some embodiments, the movable sheath 140 may be configured to house (hold) at least one therapeutic agent 150 to be delivered. The delivery device 100, including the movable sheath 140, may be configured to house and deliver any number and size of therapeutic agent. The dimensions of the movable sheath 140 may be configured according to the type and amount of the therapeutic agent 150 to be housed and delivered. In some embodiments, the second section 144 may be configured to house the therapeutic agent 150. As shown in FIG. 5, the second section 144 may form an inner cavity 146 configured to surround or to house the therapeutic agent 150.

In some embodiments, the therapeutic agent 150 may be encapsulated. Examples of encapsulation may include cells, hollow like porous spheres made of alginate, or drugs contained in nanotubes. The therapeutic agent 150 is not limited to encapsulation within spheres as shown in the figures, and may also include any known vehicle to deliver a therapeutic agent. For example, in other embodiments, the therapeutic agent 150 may be therapeutic substances suspended in a highly viscous solution, suspended in a high-volume-fraction solution, embedded in strings or filaments, as well as suspended or embedded in other packages.

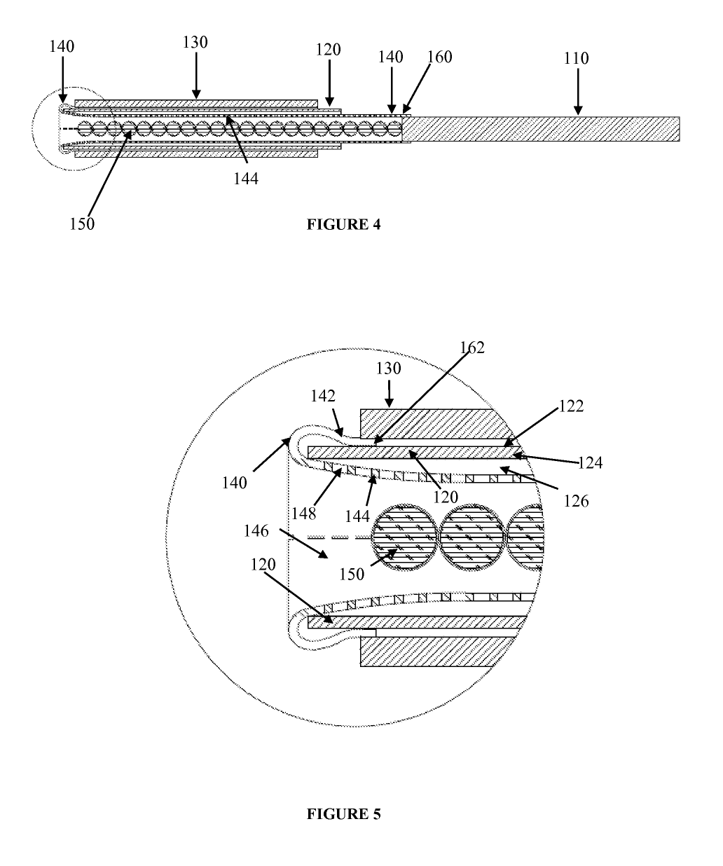

The movable sheath 140 and the push rod 110 may be fixedly disposed with respect to each other. In some embodiments, the movable sheath 140 and the push rod 110 may be fixedly attached to the each other by a fastener 160. As shown in FIG. 4, a portion of the push rod 110 at its distal end may be attached to a portion of the second section 144 of the movable sheath 140.

In some embodiments, the movable sheath 140 and the outer surface of the outer body 130 may also be, additionally or alternatively, fixedly disposed with respect to each other. In some embodiments, the movable sheath 140 and the outer body 130 may be fixedly attached to the each other by a fastener 162. As shown in FIG. 5, a portion of the second section 144 of the movable sheath 140 may be attached to the outer body 130. A portion of the movable sheath 140 may be exposed about the distal end 104 as a result of this attachment.

The fasteners 160 and 162 may be any biocompatible fastener. In some embodiments, the fasteners 160 and 162 may be a mechanical lock, such as an interference fit. In other embodiments, the fasteners 160 and 162 may be an external locking band, such as a ligature. In other embodiments, the fasteners 160 and 162 may be an adhesive. In some embodiments, the fasteners 160 and 162 may be the same fastener. In other embodiments, the fasteners 160 and 162 may be different fasteners.

The movable sheath 140 may include a plurality of perforations 148 along at least one surface. The perforations 148 may be configured to allow preferential tearing of the movable sheath 140 collinear to the axis of the delivery device 100. The perforations 148 may be formed with any known cutting means, for example, with cutting dies or ablative lasers, such as an Excimer laser. The perforations 148 may reduce hoop strength by allowing for tearing across the site of perforation during operation of the delivery device 100, for example, during retraction of the outer body 130

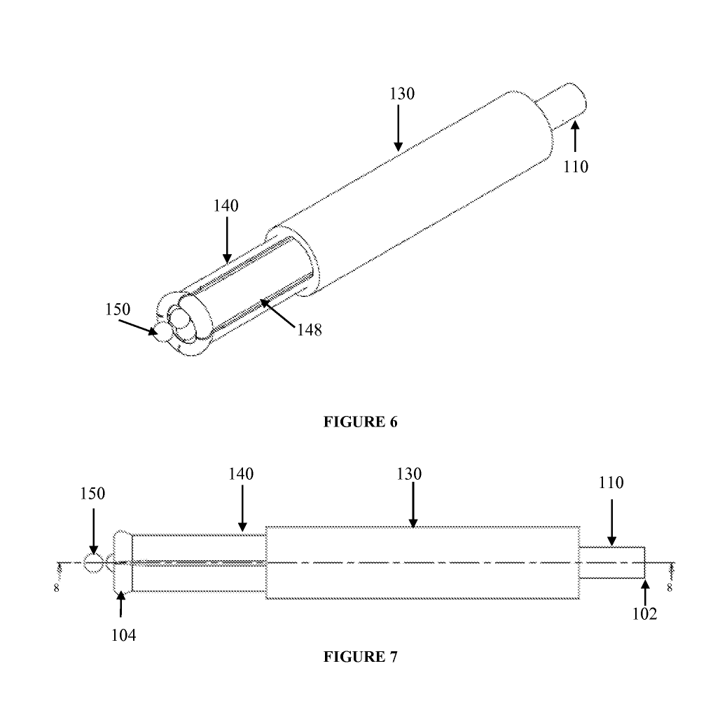

FIGS. 1-5 show the delivery device 100 in a first position. In some embodiments, the first position may be the position the delivery device 100 prior to use. FIGS. 6-11 show the delivery device 100 in two different positions (may also be referred to individually or collectively as “a second position”) during operation.

In operation, the clinician positions the distal end 104 of the delivery device 100, in the position shown in FIGS. 1 through 5, adjacent to a treatment site. Working from the proximal end 102 of the delivery device 100, the clinician may hold the main body 120 in a fixed position and retract (i.e., pull or move away from the distal end 104) the outer body 130 while advancing or pushing the push rod 110. These actions cause the movable sheath 140 to also retract or move with the outer body 130. The first section 142 of the movable sheath 140 moves away from the distal end 104 (also referred to as “a first direction”) with the outer body 130 causing the second section 144 to also advance toward the distal end 104 (also referred to as “a second direction”) with the outer body 130. The movement of the first section 142 also causes the second section 144 to invert and extend along onto a portion of the first surface 122 of the moving body 120. As the outer body 130 is moved toward the proximal end 102, the second section 144 moves over the distal end 104 onto the first surface 122 of the moving body, as shown in FIGS. 6 through 11.

As the movable sheath 140 slides over the distal end 104 onto the main body 120, the perforations 148 may cause the movable sheath 140 to split so that the movable sheath 140 may peel away and reverse direction. During this motion, the therapeutic agent 150 may be delivered or released from the distal end 104 to the treatment site.

In some embodiments, the outer body 130 may be continued to be moved toward the proximal end 102 and the push rod 120 may be advanced toward the distal end 104 until it reaches the end of the main body 120. In other embodiments, the outer body 130 may be continued to be moved toward the proximal end 102 and the push rod 120 may be advanced toward the distal end 104 until the second section 144 of the movable sheath 140 is fully retracted or extended onto the main body 120.

As shown in FIGS. 1 through 11, the delivery device 100 may be configured to allow the therapeutic agent 150 to move relative to the outer body 130 but not move relative to the movable sheath 140. The delivery device 100 thus may result in a low or zero shear loading to the therapeutic agent 150 during delivery, and may improve the ease of delivery from the contents independent of their viscosity.

In the embodiments shown in FIGS. 1 through 11, the delivery device 100 may maintain the overall length of the delivery system during retraction of the movable sheath 140 because the main body 120 is immovable with respect to the delivery device 100.

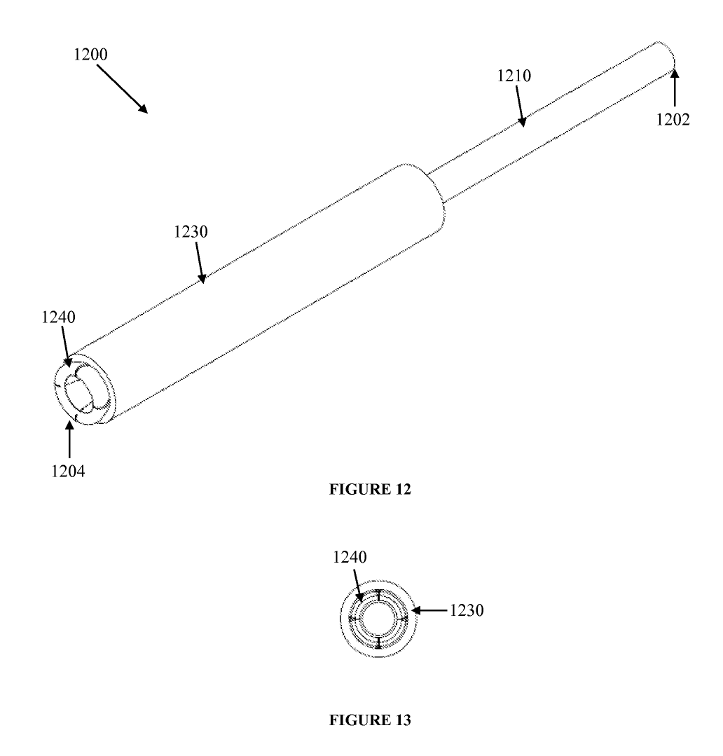

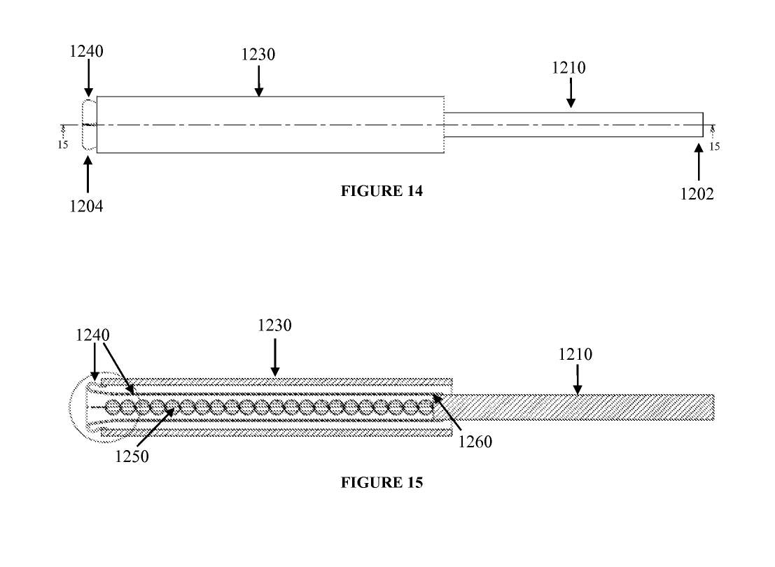

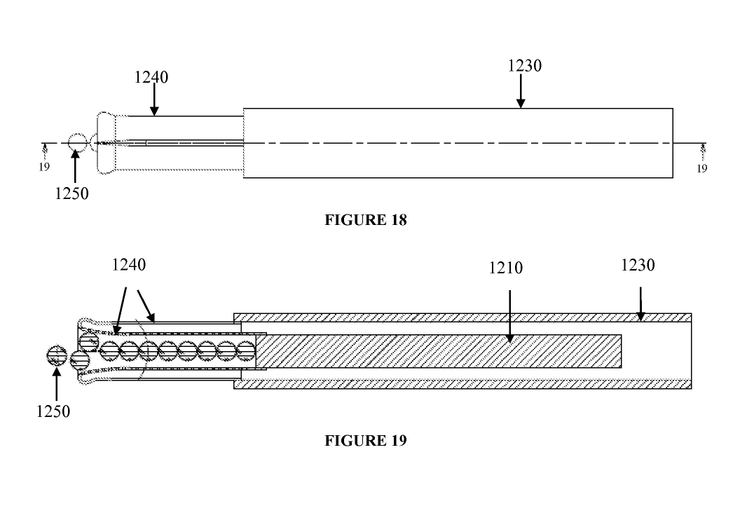

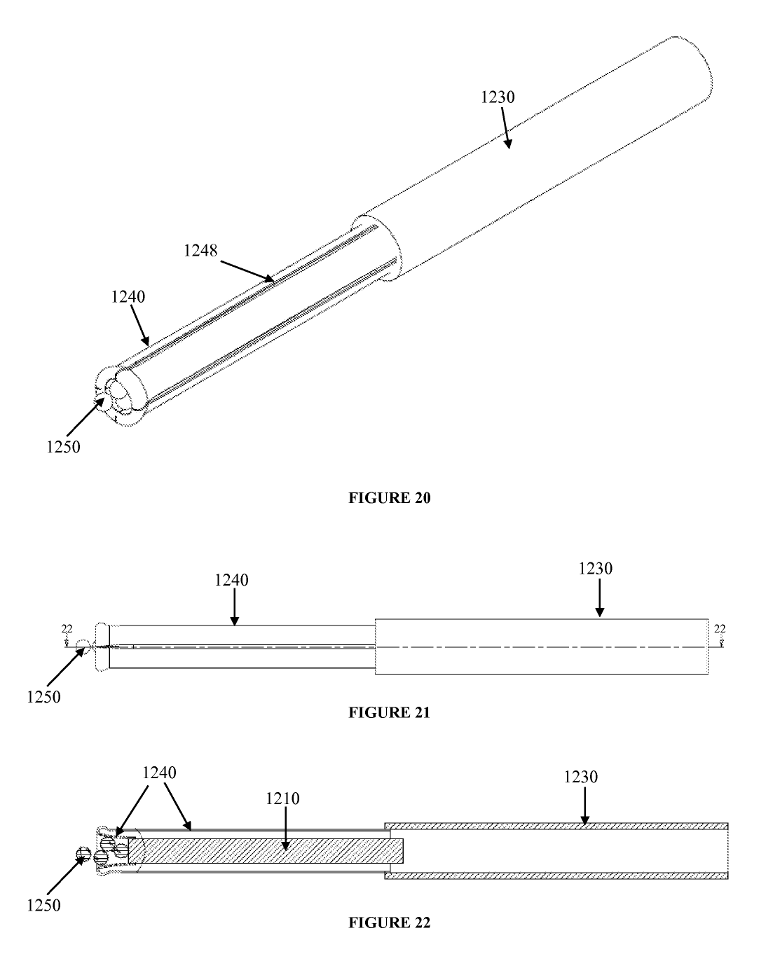

In other embodiments, a delivery device may not have the main body 120. In some embodiments, a delivery device may be configured to lay down “tracks” of a therapeutic agent. In some embodiments, a delivery device may be configured to change its length during deployment. FIGS. 12 through 22 illustrate a delivery device according to these embodiments.

As shown in FIGS. 12 through 16, a delivery device 1200 may include a fixed rod 1210, an outer body 1230, and a movable split sheath (hereinafter referred to as “movable sheath”) 1240.

The fixed rod 1210 may be a straight piece of material. The fixed rod 1210 may be any shape, including but not limited to, round or square. The diameter of the fixed rod 1210 may be mated with the inner cavity of the outer body 1230 (discussed in more detail below). The fixed rod 1210 may be configured to immovable with respect to the delivery device 1200.

The outer body 1230 may also have a tubular structure. The outer body 1230 may be concentrically arranged about the movable sheath 1240 and a portion the fixed rod 1210. The outer body 1230 may include a first surface 1232 and a second surface 1234 that opposes the first surface 1232 and borders a cavity 1236.

The fixed rod 1210 and the outer body 1230 may be made of a biocompatible high strength material. The fixed rod 1210 and the outer body 1230 may be made of the same or different material. In some embodiments, the material may be surgical stainless steel, for example, 300 series. In other embodiments, the material may be liquid crystal polymer tubing.

The movable sheath 1240 may be disposed about a distal end 1204 of the delivery device 1200 and within a length of the outer body 1230 toward a proximal end 1202 of the delivery device 1200.

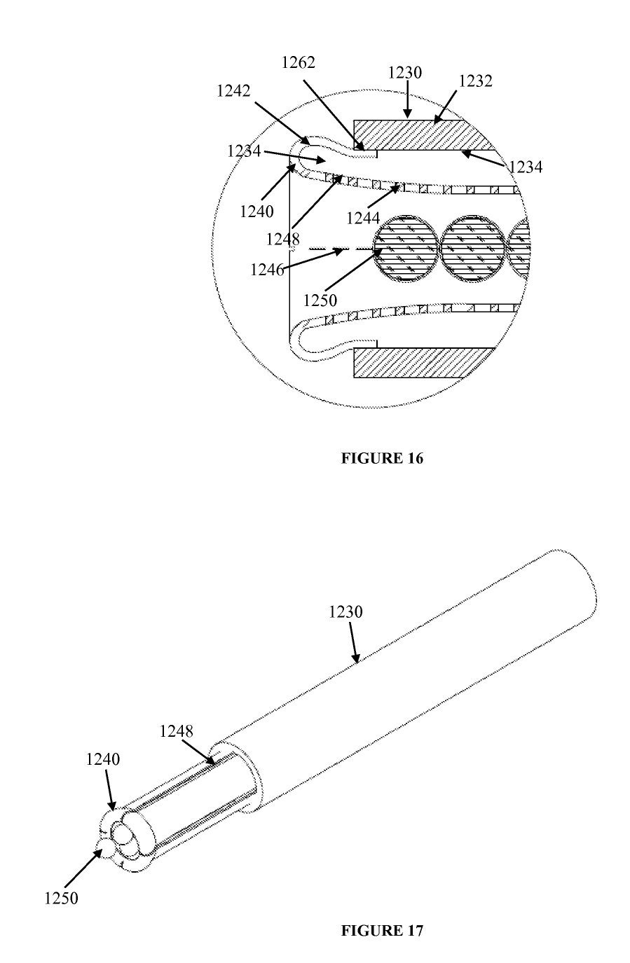

The movable sheath 1240 may be a circular surface. In some embodiments, the movable sheath 1240 may be like the movable sheath 140. At least one surface of the movable sheath 1240 may be configured to invert. The movable sheath 1240 may include a first section 1242 and an overlapping or opposing second section 1244. The second section may 1244 may extend from a portion of the fixed rod 1210 along of the length of the outer body 1230 through a distal end 1204 of the delivery device 1200. In some embodiments, the first section 1242 and the second section 1244 may meet to form the distal end 1204 of the delivery device, as shown in FIG. 16.

In some embodiments, the movable sheath 1240 may be like the movable sheath 140. The movable sheath 1240 may be made of a biocompatible material having a low surface coefficient of friction. In some embodiments, the movable sheath 1240 may be made of a plastic having a low surface of coefficient of friction. Examples of such a plastic include, but are not limited to, polytetrafluoroethylene (also known as Teflon®), fluorinated ethylene propylene (FEP), and perfluoroalkoxy (PFA). In other embodiments, the movable sheath may be made of polyethylene.

In some embodiments, the movable sheath 1240 may be configured to house (hold) at least one therapeutic agent 1250 to be delivered. The delivery device 1200, including the movable sheath 1240, may be configured to house and deliver any number and size of therapeutic agent. The dimensions of the movable sheath 1240 may be configured according to the type and the amount of the therapeutic agent 1250 to be housed and delivered. In some embodiments, the second section 1244 may be configured to house the therapeutic agent 150. As shown in FIG. 16, the second section 1244 may form and surround an inner cavity 1246 configured to house the therapeutic agent 1250.

In some embodiments, the therapeutic agent 1250 may be the same as the therapeutic agent 150. The therapeutic agent 1250 may be encapsulated. Examples of encapsulation may include cells, hollow like, porous spheres made of alginate, or drugs contained in nanotubes. The therapeutic agent 1250 is not limited to encapsulation within spheres as shown in the figures, and may also include any known vehicle to deliver therapeutic agent. For example, in other embodiments, the therapeutic agent 1250 may be therapeutic substances suspended in a highly viscous solution, suspended in a high-volume-fraction solution, embedded in strings or filaments, as well as suspended or embedded in other packages.

The fixed rod 1210 and the movable sheath 1240 may be fixedly disposed with respect to each other. In some embodiments, the movable sheath 1240 and the fixed rod 1210 may be fixedly attached to the each other by a fastener 1260. As shown in FIG. 16, a portion of the fixed rod 1210 at its distal end may be attached to the second section 1244 of the movable sheath 1240.

The movable sheath 1240 and the outer surface of the outer body 1230 may also be, additionally or alternatively, fixedly disposed with respect to each other. In some embodiments, the movable sheath 1240 and the outer body 1230 may be fixedly attached to the each other by a fastener 1262. As shown in FIG. 16, a portion of the second section 1244 of the movable sheath 1240 may be attached to the outer body 1230. This attachment may cause the first section 1242 and the second section 1244 of the movable sheath 1240 to form the distal end 1204.

The fasteners 1260 and 1262 may be any biocompatible fastener. In some embodiments, the fasteners 1260 and 1262 may be a mechanical lock, such as an interference fit. In other embodiments, the fasteners 1260 and 1262 may be an external locking band, such as a ligature. In other embodiments, the fasteners 1260 and 1262 may be an adhesive. In some embodiments, the fasteners 1260 and 1262 may be the same fastener. In other embodiments, the fasteners 1260 and 1262 may be different fasteners.

In some embodiments, like the movable sheath 140, the movable sheath 1240 may include a plurality of perforations 1248 along at least one surface. The perforations 1248 may be configured to allow preferential tearing of the movable sheath 140 collinear to the axis of the delivery device 1200. The perforations 1248 may be formed with any known cutting means, for example, with cutting dies or ablative lasers, such as a Excimer laser. The perforations 1248 may reduce hoop strength by allowing for tearing across the site of perforation during operation of the delivery device 1200, for example, during retraction of the outer body 1230

FIGS. 12 through 16 show the delivery device 1200 in a first position. In some embodiments, the first position may be the position of the delivery device 100 prior to use and loaded with a therapeutic agent. FIGS. 17 through 22 show the delivery device 1200 in different positions during operation.

In operation, the clinician places the distal end 1204 of the delivery device 1200, in the position shown in FIGS. 12 through 16, adjacent to a treatment site. Working from the proximal end 1202 of the delivery device 100, the clinician may hold the fixed rod 1210 in a fixed position and retract (i.e., pull or move away from the distal end 1204 toward the proximal end 1202) the outer body 1230. These actions cause the movable sheath 1240 to also retract or move with the outer body 130. The first section 1242 of the movable sheath 1240 moves away from the distal end 1204 (also referred to as “a first direction”) with the outer body 1230 causing the second section 1244 to also advance toward the distal end 1204 (also referred to as “a second direction”) with the outer body 1230. The movement of the first section 1242 also causes a (first) portion of the second section 1244 to invert or fold onto the other (second) portion of the second section 1244. As the outer body 1230 is moved toward the proximal end 1202, the first section 1242 expands over the second section 1244, as shown in FIGS. 17 through 22. This movement thereby exposes the movable sheath 1240 and also expands the overlap (amount of fold) between the first section 1242 and the second section 1244, as shown in FIGS. 17 through 22.

As the movable sheath 1240 is pulled by the outer body 1230 toward the distal end 1204, the perforations 1242 cause the movable sheath 1240 to split so that the movable sheath 1240 may peel away and move in a reverse direction, i.e., toward the proximal end 1202. During this motion, the therapeutic agent 1250 may be delivered or released from the distal end 1204 to the treatment site. Also, as shown in FIGS. 12 through 22, delivering the track of therapeutic agent 1250 into a treatment site causes the overall length of the delivery device 1200 to shorten or decrease.

In some embodiments, the outer body 1230 may be continued to be moved toward the proximal end 1202 until it reaches the end of the fixed rod 1210. In other embodiments, the outer body 1230 may be continued to be moved toward the proximal end 1202 until the movable sheath 1240 is fully retracted or extended onto itself

As shown in FIGS. 12 through 22, the delivery device 1200 may be configured to allow the therapeutic agent 1250 to move relative to the outer body 1230 but not move relative to the movable sheath 1240. The delivery device 1200 thus may achieve a delivery of the therapeutic agent 1250 with a low or zero shear loading, and may improve the ease of delivery from the contents independent of their viscosity.

The advantages of the disclosed delivery devices are that the relative position of the therapeutic agent to the inside [contacting] surface of the delivery devices does not change. The movable sheath maintains constant contact with the therapeutic agent as the movable sheath moves. As such, highly viscous solutions or conglomerations of therapeutic “beads” may be deployed externally to the delivery system with minimal effort for the clinician or minimized discomfort for the patient. In addition, the resulting delivery may have a low or zero “shear” event for the agent, minimizing negative affects to therapeutic efficacy.

In some embodiments, the delivery device may be sterilized. In further embodiments, the delivery device may be a single, use device. In further embodiments, the delivery device may be disposable. In some embodiments, the delivery device may be preloaded with the therapeutic agent. In other embodiments, the agent may be loaded into the delivery device via a cartridge.

All references cited herein are hereby incorporated by reference in their entirety.

While the disclosure has been described in detail with reference to exemplary embodiments, those skilled in the art will appreciate that various modifications and substitutions can be made thereto without departing from the spirit and scope of the disclosure as set forth in the appended claims. For example, elements and/or features of different exemplary embodiments may be combined with each other and/or substituted for each other within the scope of this disclosure and appended claims.

What is claimed is:

1. A delivery device for delivering a therapeutic agent from a distal end, comprising:
an outer body being configured to move away from the distal end; and
a movable sheath having a surface that includes a first section and a second section,
wherein:
the first section overlaps a portion of the second section;
the second section surrounds an inner cavity configured to hold a therapeutic agent; and
the movable sheath is configured to deliver the therapeutic agent by increasing the first section by moving the outer body away from the distal end.

2. The delivery device according to claim 1, wherein an end of the movable sheath is fixedly attached to a portion of the outer body.

3. The delivery device according to claim 1, wherein the movable sheath is configured to form the distal end.

4. The delivery device according to claim 1, wherein the movable sheath is configured to extend about the distal end.

5. The delivery device according to claim 1, wherein the movable sheath is configured to deliver the therapeutic agent by increasing the first section by inverting a portion of the second section.

6. The delivery device according to claim 5, wherein:
the movable sheath includes a plurality of perforations; and
the perforations are configured to split when the first section is increased.

7. A delivery device for delivering a therapeutic agent from a distal end, comprising:
a push rod that is configured to move toward the distal end;
an outer body that is configured to move away from the distal end; and
a movable sheath that includes a first section and a second section that opposes a portion of the first section, the second section surrounding an inner cavity configured to hold and deliver the therapeutic agent,
wherein the outer body is concentrically arranged about the second section.

8. The delivery device according to claim 7, further comprising:
a main body concentrically disposed between the outer body and the movable sheath, the main body extending between the outer body and the push rod.

9. The delivery device according to claim 8, wherein the outer body and the push rod are configured to move with respect to the main body.

10. The delivery device according to claim 7, wherein the movable sheath is configured to deliver the therapeutic agent by moving the push rod toward the distal end and the outer body away from the distal end.

11. The delivery device according to claim 9, wherein the main body includes a first surface and a second surface that opposes the first surface and borders a cavity, the first section extending along the first surface and the second section extending along the second surface.

12. The delivery device according to claim 11, wherein the movable sheath is configured to increase the first section and decrease the second section when the outer body and the push rod are moved.

13. The delivery device according to claim 8, wherein the movable sheath has a first end and a second end, the first end being fixedly disposed to an end of the push rod and the second end being fixedly disposed to an end of the outer body.

14. A delivery device for delivering a therapeutic agent, comprising:
a movable sheath having a surface, the surface includes a first section and a second section that overlaps a portion of the first section, the second section surrounding an inner cavity configured to hold and deliver the therapeutic agent;
a fixed rod; and
an outer body concentrically arranged about a portion of the movable sheath and the fixed rod,
wherein the outer body is configured to move toward the fixed rod.

15. The delivery device according to claim 14, wherein the movable sheath is configured to deliver the therapeutic agent by moving the outer body away from the distal end.

16. The delivery device according to claim 15, wherein the movable sheath is configured to increase an overlap between the first section and the second section when the outer body is moved.

17. The delivery device according to claim 15, wherein the movable sheath is configured to deliver a therapeutic agent when the first section is increased.

18. The delivery device according to claim 15, wherein the surface of the movable sheath is configured to move in a first direction and a second direction that is opposite of the first direction.

19. The delivery device according to claim 18, wherein the first direction is toward the distal end, and the movable sheath is configured to deliver a therapeutic agent when the surface changes from the first direction to the second direction.

20. The delivery device according to claim 15, wherein the outer body is configured to expose the movable sheath when the outer body is moved away from the distal end.
